(19) United States
(12) Plant Patent Application Publication
(10) Pub. No.: US 2009/0038042 P1
(43) Pub. Date: Feb. 5, 2009

(54) Loropetalum chinense variety rubrum plant named 'shang-lo'

(75) Inventors: Thomas Dennis Meadows JR., Loxley, AL (US); James Bryan Berry, Grand Saline, TX (US)

Correspondence Address: BREINER & BREINER, L.L.C., P.O. BOX 320160, ALEXANDRIA, VA, 22320-0160, US

(73) Assignee: PLANT DEVELOPMENT SERVICES, INC., Loxley, AL (US)

(21) Appl. No.: 11/878,203

(22) Filed: Jul. 23, 2007

(51) Int. Cl. A01H 5/00

(52) U.S. Cl. PLT/263.1

(57) ABSTRACT

A new and distinct variety of Loropetalum chinense variety rubrum plant named ‘Shang-lo’, characterized by its dense, low, spreading to cascading growth habit, persistent dark purple foliage color, and abundance of dark pink flowers is disclosed.

### GENUS SPECIES VARIETY

Loropetalum chinense Variety rubrum

### VARIETAL DENOMINATION

‘Shang-lo’

### BACKGROUND OF THE INVENTION

The present invention relates to a new and distinct variety of the genus Loropetalum and a member of the Hamamelidaceae family. This new Loropetalum variety, hereinafter referred to as ‘Shang-lo’ was discovered by Thomas Dennis Meadows, Jr. and James Bryan Berry in March, 2003. ‘Shang-lo’ was found as an openly pollinated seedling of Loropetalum chinense variety rubrum (unpatented), maintained by Plant Development Services Inc. in Loxley, Ala. The value of this new cultivar lies in its dense, low, spreading to cascading growth habit, persistent dark purple foliage color, and abundance of dark pink flowers. ‘Shang-lo’ has retained many of the outstanding attributes of its parent variety, in particular its tolerance of heat and disease, which makes it adaptable to culture in most of the Sunbelt States. As with the parent variety, the plant of this invention may be advantageously employed as a specimen appointment, in either formal or informal groupings, and is quite attractive in mass plantings. ‘Shang-lo’ is adaptable for culture as a potted plant. ‘Shang-lo’ is responsive to pruning and training and may be employed in forming a dense groundcover, and maintained without an excessive amount of care. This plant is easy to care for and maintain in size due to its short internodes, heavy branching, and dense canopy. Its natural propensity to remain small to maturity makes it valuable for landscape uses in smaller home gardens which require plants that do not outgrow their intended mature dimensions.

Asexual propagation of the new plant by cuttings has been under Mr. Meadows' direction in Loxley, Ala. The new plant retains its distinctive characteristics and reproduces true to type in successive generations of asexual reproduction. The plant cannot be reproduced true from seed.

### SUMMARY OF THE INVENTION

The following are the most outstanding and distinguishing characteristics of this new cultivar when grown under normal horticultural practices in Loxley, Ala.
1. Dense, low and spreading to cascading in nature.
2. The upper surface of the mature leaf is a lustrous dark purple color throughout the year.
3. Underside of leaf is an attractive grayish purple color.
4. Moderate growth rate under normal fertilization and moisture conditions.
5. Hardy to Zone 7.
6. Tolerates full sun to part shade.
7. Heat tolerant.
8. Easily propagated with semi-hardwood cuttings in late spring through the summer.
9. Requires little pruning but is tolerant if pruning is needed.
10. Relatively pest resistant.
11. Good specimen plant.
12. Good groundcover plant.
13. Very desirable in decorative containers and hanging baskets.
14. The flowers are dark pink and profuse.

### DESCRIPTION OF THE DRAWINGS

This new Loropetalum chinense variety rubrum cultivar is illustrated by the accompanying photographic prints in which:

FIG. 1 is a photograph of a close-up showing the flower, buds, foliage and stem color, as well as flower size and form.

FIG. 2 is a photograph showing the dense, low, spreading to cascading growth habit of a three gallon plant.

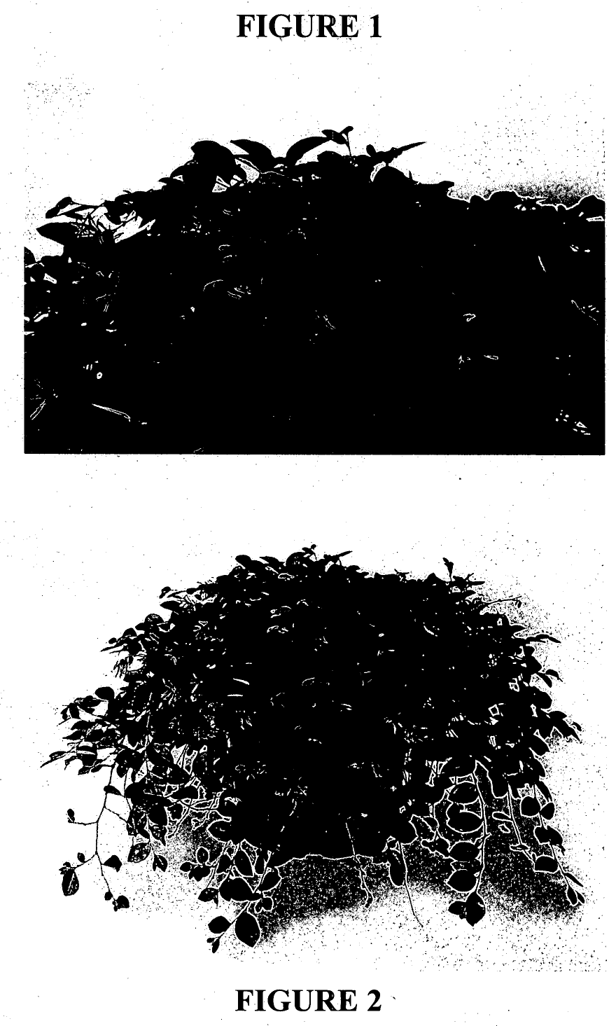

The colors shown are as true as is reasonably possible to obtain by conventional photographic procedures. Colors in the photographs may appear different than actual colors due to light reflectance. The colors of the various plant parts are defined with reference to The Royal Horticultural Society Colour Chart. Description of colors in ordinary terms are presented where appropriate for clarity in meaning.

### BOTANICAL DESCRIPTION OF THE PLANT

The following is a detailed description of the new variety of Loropetalum based on observations made of two year old plants grown in three gallon containers in commercial production practices, in greenhouses, and in established landscape plantings in Loxley, Ala.

Distinctive Characteristics:

TABLE 1

| | Loropetalum | | Loropetalum | Loropetalum |
|---|---|---|---|---|
| | c. var. | Loropetalum | c. | c. ‘Sato's |
| Charac- | rubrum | c. Var. | ‘Shang-hi’ | Dwarf Red’ |
| teristic | ‘Shang-lo’ | rubrum | PPAF | PP#17669 |
| Height | 1-2 feet | 10-12 feet | 4-5 feet | 1.97 feet |
| (Mature) | (30.5-61 cm) | (3.0-3.7 m) | (1.2-1.5 m) | (60 cm) |
| Width | 4-5 feet | 8-9 feet | 3-4 feet | 5.91 feet |
| (Mature) | (1.2-1.5 m) | (2.4-2.7 m) | (0.9-1.2 m) | (180 cm) |
| Leaf | 9/16\-1⅛\ | 1½\-2¼\ | ⅞\-1⅜\ | 0.39-0.98 |
| Length | inches | inches | inches | inches |
| | (1.4-2.9 cm) | (3.8-5.7 cm) | (2.2-3.5 cm) | (1-2.5 cm) |
| Leaf Width | 7/16\-⅞\ | ¾\-1⅛\ | ⅝\-⅞\ | 0.39-0.59 |
| | inches | inches | inches | inches |
| | (1.1-2.2 cm) | (1.9-2.9 cm) | (1.6-2.2 cm) | (1-1.5 cm) |
| Growth | Dense, low, | Upright, | Dense, | Prostrate |
| Habit | spreading to | Spreading | upright, | to mounding |
| | cascading | | globose | |
| Foliage | Greyed- | Yellow-Green | Greyed- | Near |
| Color | Purple G. | G. 147A | Purple G. | Greyed- |
| (Immature) | 187B | overlain | 187B | Purple G |
| | | with Greyed- | | 185A-B |
| | | Purple G. | | |
| | | 187B | | |
| Foliage | Greyed- | Greyed- | Greyed- | Near |
| Color | Purple G. | Purple G. | Green | Greyed- |
| (Mature) | 187A | 187A to | G. 187A | Purple G |
| | | Green G. | | 183A-B |
| | | 139A | | |
| Flower | Red-Purple | Red-Purple | Red-Purple | Red G. 55B |
| Color | G. 61B | G. 67B | G. 61B | to 51A, |
| | | | | 53C, 52A |
| | | | | and 53D |
| Leaf Shape | Elliptical | Ovate | Elliptical | Ovate |

The female, or seed parent, of ‘Shang-lo’ is Loropetalum chinense variety rubrum (unpatented) which is a native of China, and was first discovered in 1928 in Hunan Province. It was introduced to the United States in the 1980's.

Loropetalum chinense variety rubrum (unpatented), Loropetalum chinense variety rubrum ‘Shang-hi’ (U.S. Plant patent application Ser. No. 11/488,651), and Loropetalum chinense variety rubrum ‘Sato's Dwarf Red’ (U.S. Plant Pat. No. 17,669) are well known in the industry and are comparable to ‘Shang-lo’ in that all have reddish purple foliage color at some point during the year. However, there are many differences. The foliage of ‘Shang-lo’ is slightly smaller than ‘Shang-hi’ or the parent variety. The mature height of ‘Shang-lo’ is much lower than ‘Shang-hi’ or the parent variety. The flower color of ‘Shang-lo’ is darker than the parent variety and more purple than ‘Sato's Dwarf Red’. The immature and mature foliage colors of ‘Shang-lo’ are a darker purple than those of ‘Sato's Dwarf Red’.
Classification:
Botanical: Loropetalum chinense variety rubrum ‘Shang-lo’
Parentage: Openly pollinated seedling of Loropetalum chinense variety rubrum
Commercial: Broadleaf evergreen.
Form: Dense, low, spreading to cascading
Height: 1-2 feet (30.5-61 cm)
Width: 4-5 feet (1.2-1.5 m)
Growth Rate: Moderate under normal fertilization and moisture conditions. Semi-hardwood cuttings taken in late spring and through the summer produce rooted cuttings in four to five months in Loxley, Ala. Root development is vigorous and finely branched. In a period of four years from a rooted cutting, the plant reaches a height of 10 inches (25.4 cm) and a spread of 3 feet (0.9 m) under normal growing conditions in Loxley, Ala. Young limbs tend to grow upward and then outward and eventually cascade downward. The plant normally grows at the rate of about 6 inches (15.24 cm) or more per year and reaches a height of 2 feet (61 cm) and a spread of 5 feet (1.5 m) at maturity while maintaining a dense habit due to the abundant branch development.
Foliage: Alternate, simple, evergreen, elliptic, sparsely stellate-pubescent, coriaceous, almost flat, and varying in size from 9/16 inch (1.4 cm) to 1 ⅛ inches (2.9 cm) long and 7/16 inch (1.1 cm) to ⅞ inch (2.2 cm) wide. The petioles are ⅛ inch (0.3 cm) to 3/16 inch (0.5 cm) long, 1/32 inch (0.08 cm) in diameter, heavily stellate-pubescent and Greyed-Purple Group 187B. The venation pattern is pinnate. The base of the leaf is oblique, the margin entire, and the apex acute. The upper surface of the immature leaf is Greyed-Purple Group 187B and the underside is Purple Group 79D underlain with Greyed-Purple Group 187B. Immature midribs and veins are depressed on the upper side (Greyed-Purple Group 187B) and prominent on the underside (Purple Group 79D). These immature leaf colors last three to four weeks in Loxley, Ala. The upper surface of the mature leaf is closest to but darker than Greyed-Purple Group 187A with closest to but darker than Yellow-Green Group 147A undertones. Mature midribs and veins are depressed on the upper side (Greyed-Purple Group 187A) and prominent on the under side (Greyed-Green Group 194C). These mature leaf colors are persistent throughout the winter. The paired foliaceous stipules are 1/16 inch (0.16 cm) to ⅛ inch (0.3 cm) long and 1/32 inch (0.08 cm) to 1/16 inch (0.16 cm) wide. The upper surface and under surface are Greyed-Purple Group 187A. The stipules are caducous. In 2005, the date of initial spring growth was March 10, in Loxley, Ala. After the initial spring flush there was almost continuous slow growth until fall, ending October 28, also in Loxley, Ala. When grown in full sun, the internode length of this plant is ¼ inch (0.6 cm) to ½ inch (1.3 cm). When grown in light shade the internode length is 5/16 inch (0.8 cm) to ¾ inch (1.9 cm). As would be expected, a plant grown in the shade results in a taller, less dense plant with larger leaves.
Stems: The young stems are Greyed-Purple Group 187A and heavily stellate-pubescent. During the second growing season they become Brown Group 200C. The pith is solid and uniform. Young and older stems are densely branched.
Flowers: Perfect, Red-Purple Group 61A (upper surface and under surface), glabrous, four strap-shaped petals. The flowers form a loose funnel, are borne on current season's growth, and are non-fragrant; they last on the plant in the garden five to seven days. Tight buds at 3/16 inch (0.5 cm) are orbicular, Red-Purple Group 63B and pubescent. Six to eight flowers form globose heads on branchlet terminals and occasionally one to two flowers in the leaf axils. Flowering occurs from early March to late March with light repeat blooming throughout the growing season. These racemes are 1 inch (2.5 cm) to 1 5/16 inches (3.3 cm) in diameter by ½ inch (1.3 cm) to ¾ inch (1.9 cm) in depth and abundantly cover the plant. The flowers are attached in a circular pattern to short peduncles which are pubescent, 1/16 inch (0.2 cm) long, 1/32 inch (0.08 cm) in diameter and Red-Purple Group 63B. The four petals which arise from the calyx tube at the sepal base are linear, crinkled, ½ inch (1.3 cm) to ¾ inch (1.9 cm) long, 1/16 inch (0.2 cm) to ⅛ inch (0.3 cm) wide, with retuse apexes and entire margins. There are four stamens 1/32 inch (0.08 cm) long, Red-Purple Group 63A, with anthers also Red-Purple Group 63A. Pollen matures to Yellow-Green Group 11D and is produced in very small amounts. The two pistils are 1/32 inch (0.08 cm) long and Red-Purple Group 63A. The ovary is inferior and two-celled. Each flower has four sepals that have entire revolute margins which are 1/16 inch (0.2 cm) long, 1/16 inch (0.2 cm) wide, ovate, and fused into a calyx. The calyx is 3/16 inch (0.5 cm) in diameter, 3/16 inch (0.5 cm) in depth, pubescent and Red-Purple Group 63B outer surface and Red-Purple Group 63A inner surface.
Fruit: Woody, scabrous, ovoid, 3/16 inch (0.5 cm) to 5/16 inch (0.8 cm) long, ⅛ inch (0.3 cm) to ¼ inch (0.6 cm) in diameter, two-celled capsule, one seed per cell. Mature plants produce 75 to 100 fruits which are Yellow-Green Group 146B. Seeds are ⅛ inch (0.3 cm) to 3/16 inch (0.5 cm) long, 1/16 inch (0.2 cm) to ⅛ inch (0.3 cm) in diameter, Brown Group 200C, and mature in the fall.
Culture: Grows well in a wide range of conditions and tolerates sun to part shade. Prefers a moist, well-drained soil that is rich in organic matter. Responds well to mulching and medium applications of fertilizer; prefers pH 6 to 7. Very little pruning is needed. Adaptable to containers, above ground planters, and hanging baskets. Makes a good, dense groundcover with excellent foliage and flower contrast. Ideal for coastal regions and warmer parts of the Piedmont. Cold hardiness and drought resistance are comparable to the parent variety. Propagated with semi-hardwood cuttings in late spring through the summer.
Disease and insect resistance: Resistance to disease and insects common to plants of Loropetalum has not been observed. The exemplary embodiments herein disclosed are not intended to be exhaustive or to unnecessarily limit the scope of the invention. The exemplary embodiments were chosen and described in order to explain the principles of the present invention so that others skilled in the art may practice the invention. As will be apparent to one skilled in the art, various modifications can be made within the scope of the aforesaid description. Such modifications being within the ability of one skilled in the art form a part of the present invention and are embraced by the appended claim.

It is claimed:

1. A new and distinct variety of Loropetalum chinense variety rubrum plant named ‘Shang-lo’ as illustrated and described.
